(19) United States
(12) Patent Application Publication
(10) Pub. No.: US 2004/0248284 A1
(43) Pub. Date: Dec. 9, 2004

(54) System and method for analyzing a blood sample and disposable cartridge for use in this system or method

(76) Inventor: Marinus Gerardus Johannes van Beuningen, Oss (NL)

Correspondence Address: AMSTER, ROTHSTEIN & EBENSTEIN, 90 PARK AVENUE, NEW YORK, NY, 10016

(21) Appl. No.: 10/497,934

(22) PCT Filed: Dec. 3, 2002
(86) PCT No.: PCT/EP02/13734

(30) Foreign Application Priority Data
Dec. 6, 2001 (EP) 01204778.3

(51) Int. Cl. C12Q 1/68; C12M 3/00

(52) U.S. Cl. 435/287.2; 435/6

(57) ABSTRACT

A system for analyzing a blood sample comprises an element for nucleic acid (NA) isolation, an element for NA amplification, an incubator and a detector for detecting a parameter of the blood sample. The system comprises a single disposable cartridge having the NA isolation element and the NA amplification element, and a device having the incubator and the detector. The device is provided with a receiving space and each cartridge is adapted to be inserted into the receiving space. The NA amplification part of the cartridge is located within the incubator when the cartridge has been inserted into the receiving space.

The invention relates to a system for analyzing a blood sample, comprising an element for nucleic acid (NA) isolation, an element for NA amplification, an incubator and a detector for detecting a parameter of the blood sample, and to a method for analyzing a blood sample of a patient, comprising lysis of the blood sample to isolate NA, amplifying the isolated NA, binding the amplified NA with a fluorescent probe and detecting fluorescent emission. The invention further relates to a disposable cartridge for use in this system or method.

Such systems and methods are known and are used for example to test a blood sample for the presence of a HIV virus. Generally such a system is used in a laboratory environment, and the method is carried out by laboratory staff. The known system requires a patient to visit a hospital or the like to have his/her blood tested. In order to conveniently collect a blood sample, EP-A-0 717 283 discloses a collection device which can be used by a patient to collect a blood sample. However in this known device, the patient still has to submit the collection device to a laboratory for subsequent analysis still requiring involvement of laboratory staff.

The object of the invention is to provide a system and method of the above-mentioned type which can be used in an easy manner outside a laboratory and the use of which does not require any skilled staff.

According to the invention the system is characterized by a disposable cartridge having the NA isolation element and the NA amplification element, and a device having the incubator and the detector, wherein the device is provided with a receiving space and each cartridge is adapted to be inserted into the receiving space, wherein the NA amplification part of the cartridge is located within the incubator when the cartridge has been inserted into the receiving space.

In this manner a system is provided, wherein the complete treatment of the blood sample for analysis purposes is carried out within a single disposable cartridge, so that the use of the system does not require laboratory circumstances or skilled laboratory staff.

Accordingly the method of the invention is characterized in that the steps of lysis of the blood sample to isolate NA, amplifying the isolated NA, and binding the amplified NA with a fluorescent probe are carried out in a single disposable cartridge comprising an lysis chamber and an amplification chamber.

Further the invention provides a disposable cartridge for use in the system or method according to the invention, comprises a lysis chamber, an amplification chamber and a transfer element for transferring a fixed amount of lysed blood sample from the lysis chamber to the amplification chamber.

The invention will be further explained by reference to the drawings schematically showing an embodiment of the system of the invention.

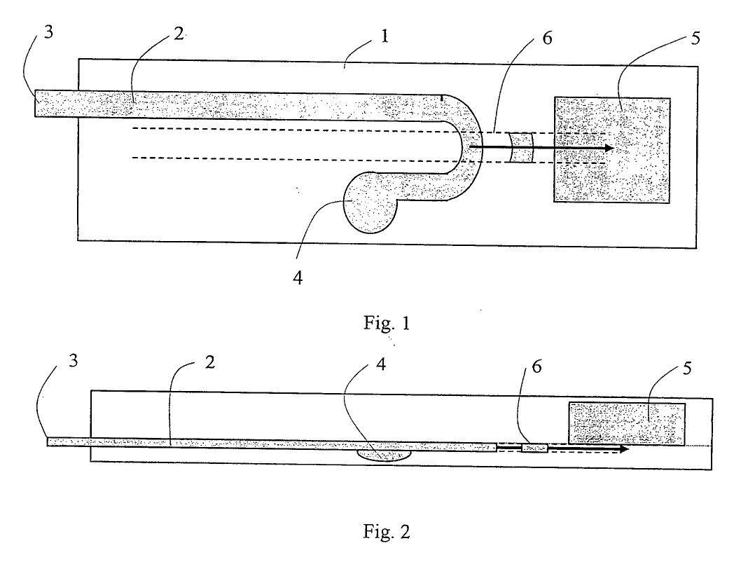

FIGS. 1 and 2 show a top view and side view, respectively of an embodiment of the cartridge of the invention.

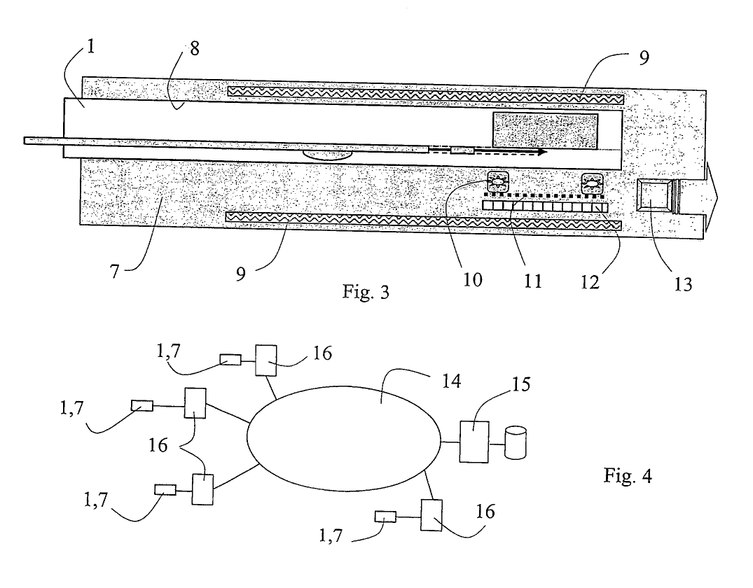

FIG. 3 shows a cross-section of an embodiment of the device of the system of the invention, wherein the cartridge of FIGS. 1 and 2 is loaded into the device.

FIG. 4 shows an embodiment of the system of the invention as used with a PC connected to the internet.

FIGS. 1 and 2 show a top and side view, respectively of a cartridge 1 which is part of a system for analyzing a blood sample of a patient. The system will be described in an embodiment which can be used for HIV quantitation in order to determine the viral load of the blood sample. In this manner the efficacy of anti-viral therapies can be monitored. In a slightly different embodiment the system can also be used for HIV genotyping.

The cartridge 1 comprises a lysis chamber 2 which is made as a capillary having a contents of 10-50 μl, for example. The capillary 2 has an inlet 3 at one end for introducing the blood sample and an air compartment 4 at its other end. By using a capillary as lysis chamber, blood can be easily introduced by simply contacting the inlet 3 with a blood drop. In a preferred embodiment, the capillary 2 contains near the inlet 3 a reagent for lysis of the blood sample during introduction of the blood into the capillary 2. Capillary suction by the chamber 2 automatically stops as soon as the capillary is filled up to the air compartment 4.

The cartridge 1 further comprises an amplification chamber 5 and a transfer element 6 which is schematically indicated by a dashed line. The transfer element 6 is movable to move an intermediate part of the capillary 2 filled with a fixed amount of the blood sample to the amplification chamber 5. In this manner a fixed amount of blood sample can be transferred to the amplification chamber 5.

In a preferred embodiment the amplification chamber 5 contains amplification reagents, in particular reagents for amplification by means of a method known as NASBA. Further the amplification chamber 5 preferably contains so-called fluorescent probes such as molecular beacons.

The system further comprises a device 7 schematically shown in cross section in FIG. 3. The device 7 is provided with a receiving space 8 for receiving the cartridge 1. The device 7 comprises in this embodiment two heating elements 9 with a temperature control circuit not further shown. In this manner an incubator is obtained for maintaining the amplification chamber 5 at an amplification temperature of 41° C. As an alternative for NASBA TMA, SDA or other isothermal amplification methods could be used.

In case of HIV viral load quantitation, the amplification chamber 5 also contains a control nucleic acid (NA). Further two different fluorescent probes are used in this case, one probe binding to the control NA and the other probe binding to the NA in the blood sample to be analyzed. With respect to the chemistry behind the test reference is made to an article “Development of a high throughput detection system for HIV-1 using real-time NASBA based on molecular beacons”, by R. van Beuningen et al., Proceedings of SPIE 4264, pages 66-71.

The device is provided with a detection system including one or more light emitting diodes (LED's) 10, an optical filter 11 and an array of photodiodes or a CCD camera 12 as optical transducer. The output signal of the transducer 12 is delivered to an electronic circuit 13 with an interface adapted to be connected to an input port of a PC. This PC can be programmed to analyze the information on the blood sample received from the device. Further this PC comprises a patient database for storing patent data including parameter information on the patient's blood. By comparing the parameter information of the blood sample under test with the stored parameter information a change in the viral load of the blood sample can be detected. In this manner the parameter information in the database can be used to predict disease progression and to give information to the user on how to use the antiviral medication treatment. Further the PC can advise to consult a doctor for a new therapy regimen.

As an alternative to a connection to a PC programmed in a manner as described above, the PC can be connected through the internet 14 to a server 15 as shown in FIG. 4. As shown a number of PC's 16 can communicate with the server 15. The device 7 with a cartridge 1 is connected to the PC 16. The server 15 will be programmed as described above. In this manner a number of patients can use the device and disposable cartridges as described in a simple manner, wherein the parameter information obtained is forwarded to the server 15 for analysis using a patient database as schematically indicated.

In the above-described embodiment the cartridge 1 is adapted for use in a HIV viral load quantitation test. A slightly different cartridge with the device as described can be used for HIV genotyping. In this case the amplification chamber 5 of the cartridge will be provided with an array of binding areas, wherein each area contains a different binding substance. The optical information described can be analyzed as described in an international patent application PCT/EP01/08012 of the same applicant.

In summary the system can be used to analyze a blood sample of a patient comprising the steps of lysis of the blood sample to isolate NA, amplifying the isolated NA, binding the amplified NA with a fluorescent probe and detecting fluorescent emission. All chemistry takes place within a single disposable cartridge comprising a lysis chamber and an amplification chamber for the amplification, the amplification reagents can either be pre-stored in the amplification chamber or can be added to the amplification chamber from a separate amplification reagents storage compartment. This storage compartment can be incorporated in the cartridge 1 or can be delivered as a separate part. The same applies to a control NA and the fluorescent probe substances. In such an embodiment the cartridge and storage parts can be provided together with an instruction booklet in a complete kit for a HIV viral load quantitation test or a HIV genotyping test.

In use, the device is connected to an input port of a PC and in the embodiment of FIG. 4 a web browser is used for login on the web database running on the server. In a usual manner user verification and preferably device verification is carried out. A new disposable cartridge is loaded into the receiving space of the device. The sample area, including the amplification chamber 5 is pre-warmed to amplification temperature. If amplification reagents are not present in the amplification chamber 5, these reagents are added to the chamber 5. The patient pricks his/her finger to draw some blood and the blood is taken up in the capillary of the cartridge 1. During introduction lysis of the blood sample takes place at the first part of the capillary 2. A fixed amount of lysed blood sample is transferred by the transfer element 6 to the amplification chamber 5. Amplification and real-time detection using fluorescent emission from the fluorescent probes are carried out by the optical system of the device. The data obtained in this manner is transferred to the PC and forwarded by the PC to the database running on the server. The server is programmed to interpret the data as received and the results of the data analysis are stored in the web database. The cartridge 1 can be disposed after use.

The invention is not restricted to the above-described embodiments which can be varied in a number of ways within the scope of the invention.

1. A system for analyzing a blood sample, comprising an nucleic acid (NA) isolation element, an NA amplification element comprising an amplification chamber, an incubator and a detector for detecting a parameter of the blood sample, characterized by a disposable cartridge having the NA isolation element and the NA amplification element, and a device having the incubator and the detector, wherein the device is provided with a receiving space and each cartridge is adapted to be inserted into the receiving space, wherein the amplification chamber is located within the incubator when the cartridge has been inserted into the receiving space.

2. The system according to claim 1, wherein the NA isolation element of the cartridge is provided with a lysis chamber containing a reagent capable of lysing a blood sample, the chamber having an inlet for introducing a blood sample, wherein the cartridge further comprises a transfer element for transferring a fixed amount of lysed blood sample to the amplification chamber.

3. The system according to claim 2, wherein the lysis chamber is a capillary having the inlet at one end and an air compartment at another end, wherein the transfer element comprises an intermediate part of the capillary, which intermediate part is movable within the cartridge to transfer said fixed amount of lysed blood sample to the amplification element.

4. The system according to claim 1, wherein the NA amplification element contains an amplification reagent.

5. The system according to claim 1, wherein the NA amplification element contains an array of binding areas, each area having a different binding substance.

6. The system according to claim 1, wherein the NA amplification element contains at least one fluorescent probe.

7. The system according to claim 1, wherein the detector of said device comprises at least one light emitter capable of illuminating the NA amplification element and an optical transducer capable of receiving fluorescence emission from the NA amplification element, said transducer providing an electrical signal containing information on the parameter of the blood sample.

8. The system according to claim 7, wherein said device is provided with an interface for communication with a computer to deliver the parameter information to the computer, wherein the computer is adapted to process the parameter in-formation received from the device.

9. The system according to claim 8, wherein said device is provided with an interface for connection to a PC to deliver the parameter information to the PC, wherein said PC is adapted to communicate with a central server, wherein the server comprises a database for storing patient data including parameter information of the patient's blood, wherein the server is adapted to compare the parameter information of the blood sample with the stored parameter information and/or to analyze the parameter information to determine a new parameter.

10. A method for analyzing a blood sample, comprising lysis of the blood sample to isolate NA, amplifying the isolated NA, binding the amplified NA with a fluorescent probe and detecting fluorescent emission, characterized in that the steps of lysis of the blood sample to isolate NA, amplifying the isolated NA, and binding the amplified NA with a fluorescent probe are carried out in a single disposable cartridge comprising an lysis chamber and an amplification chamber.

11. The method according to claim 10, wherein a disposable cartridge is loaded into a device comprising an incubator and a detector for detecting fluorescence emission, wherein at least the amplification chamber is heated to amplification temperature and a blood sample is introduced into the lysis chamber to start the analyzing process.

12. The method according to claim 11, wherein a fixed amount of the lysed blood sample is transferred within the cartridge from the lysis chamber to the amplification chamber to start the NA amplification, wherein the detector detects real-time the fluorescence emission, and wherein in-formation on the fluorescence detection is transferred to a computer for data analysis.

13. The method according to claim 10, wherein the blood sample is analyzed for HIV viral load quantitation.

14. The method according to claim 10, wherein the blood sample is analyzed for HIV genotyping.

15. The method according to claim 10, wherein the device is connected to a PC, wherein the PC is communicating with a central server to transfer the fluorescence information to the central server.

16. A disposable cartridge comprising a lysis chamber, an amplification chamber and a transfer element suitable for transferring a fixed amount of lysed blood sample from the lysis chamber to the amplification chamber.

17. The disposable cartridge according to claim 16, wherein the lysis chamber contains a reagent capable of lysing a blood sample and the amplification chamber contains an amplification reagent and at least one fluorescent probe.

18. The disposable cartridge according to claim 16, wherein the lysis chamber is a capillary having at one end an inlet for introducing a blood sample and at another end an air chamber.

19. The disposable cartridge according to claim 17, wherein the amplification chamber contains a control NA and a second fluorescent probe.

20. The disposable cartridge according to claim 16, wherein the amplification chamber comprises an array of NA binding areas, each area having a different binding substance.

21. A kit comprising a cartridge according to claim 16 and at least one container with a reagent selected from the group consisting of a reagent capable of lysing a blood sample, an NA amplification reagent and a fluorescent probe.
